(19) United States
(12) Patent Application Publication
(10) Pub. No.: US 2008/0200909 A1
(43) Pub. Date: Aug. 21, 2008

(54) Laser fiber holder

(76) Inventor: David E. Lawler, Bloomington, IN (US)

Correspondence Address: David W. Carrithers;CARRITHERS LAW OFFICE, PLLC, One Parago Centre, 6060 Dutchman's Lane, Suite 140, Louisville, KY, 40205, US

(21) Appl. No.: 11/985,869

(22) Filed: Nov. 16, 2007

Related U.S. Application Data
(60) Provisional application No. 60/859,321, filed on Nov. 16, 2006.

(51) Int. Cl. A61B 18/22

(52) U.S. Cl. 606/15

(57) ABSTRACT

A laser fiber holder for exposing laser energy to living tissue, comprising a longitudinal sleeve having a translucent distal end section for encasing an extendible laser fiber tip energizable for exposing laser energy to living tissue and a proximal end having a smaller opening for cooperatively engaging the laser fiber in a friction fit holding the fiber within the sleeve at a desired length and holding the tip at a set distance invention the tissue providing laser energy at selected point above the tissue and protecting the eyes of the user from direct laser light.

This application claims priority from U.S. Provisional application Ser. No. 60/859,321 filed Nov. 16, 2006.

### FIELD OF INVENTION

The present invention relates to devices for treating periodontal disease and skin maladies, blemishes or discolorations, particularly to devices which incorporate laser energy shown on the affected area and used to either stimulate tissue healing or growth or to kill bacteria or debride diseased tissue.

### BACKGROUND OF THE INVENTION

The use of laser light for treatment of various skin or periodontal issues has been common for years. The laser beam is exposed to the skin, tissue, or a die applied to the skin or tissue to concentrate the energy at the desired target. The target is then heated by the laser energy and the desired effect is accomplished. The laser is aimed at the target by holding a handpiece containing optical fibers which transmit the laser beam to the target. The beam is emitted from the end of the handpiece and can thus be aimed at the target.

A typical example of a such a tool in use today is the Millennium TrueFlex laser fiber holder as pictured in FIG. 5. It uses a 6 watt Nd:YAG (Neodymium doped yttrium aluminum garnet) laser as the energy source. As shown in the figure, the optical fiber protrudes from the end of the hand piece and thus must be held the right distance from the target (e.g. gums or skin).

Three problems are inherent in typical devices used today:
A. While using handpiece, care must be taken not to get the fiber too close to or even to touch the target area. Further, the distance from the fiber tip to the target should be easily adjustable and easy to held at the proper distance;
B. An additional bright red light is emitted from the fiber, the purpose of which is to indicate to the user where the laser light is concentrated and the bright light can be irritating to the user; and
C. After continued use, the handpiece can become uncomfortably hot.

### DESCRIPTION OF PRIOR ART

In Edward LaBudde et al. U.S. Pat. No. 6,607,524 B1 and Vassiliadis U.S. Pat. No. 5,207,576 devices are taught which employ a handpiece containing a fiber emitting laser energy to a target area but neither employs an adjustable sheath for maintaining the safe distance between the fiber tip and the target tissue.

A tool is needed which eases and encourages proper, safe and comfortable placement of the laser energy and prevents the bright red aiming from irritating the user.

### BRIEF DESCRIPTION OF THE DRAWINGS

A better understanding of the present invention will be had upon reference to the following description in conjunction with the accompanying drawings in which like numerals refer to like parts throughout the several views and wherein:

FIG. 1 is a perspective view of the laser fiber holder showing the laser optical fiber sheath with one end inserted into the handpiece and the other end assembled to a connector for couple to a laser generator;

FIG. 2 is a perspective view of the laser fiber holder of FIG. 1 showing the handpiece disassembled with the fiber sheath still inserted into the metallic sleeve portion of the handpiece;

FIG. 3 is a perspective view of the laser fiber holder of FIG. 1 depicting the plastic sleeve portion with a threaded end;

FIG. 4 is an end view of the sleeve of the laser fiber holder of FIG. 1 showing the distal end of the plastic sleeve with the optical fiber positioned inside the sleeve;

FIG. 5 is a perspective view of the shows how the handpiece of the laser fiber holder is positioned when in use; and

FIG. 6 depicts shows a conventional prior art holder.

### SUMMARY OF THE INVENTION

The present invention comprises a laser fiber holder for exposing a laser energy to living tissue. It includes a longitudinal sleeve defining a translucent distal end sleeve portion for encasing an extendible laser fiber tip spaced apart from an interior bore sidewall thereof and energizable for biostimulation treatment of tissue. A proximal sleeve portion having a proximal end including a small opening sized for cooperatively engaging the insulated exterior surface of the laser fiber in a friction fit holding the fiber within the sleeve at a desired length and holding the tip at a set distance from the tissue providing the laser energy at a selected point above the tissue and protecting the eyes of the user from direct laser light.

Moreover, a preferred embodiment of the longitudinal sleeve comprises a first translucent section having an inner bore spaced apart from the laser tip, the first translucent section co-operatively engaging a second sleeve section having an interior bore for cooperatively and slidably engaging the laser fiber insulated exterior surface in a friction fit. The second sleeve section comprises a heat resisting material.

It is an object of the present invention to provide a laser fiber holder which eases the proper placement and delivery of the laser energy exposed to living tissue during medical procedures. The device allows for easy positioning of the fiber and thus the fiber tip remains at a fixed distance from the end of the tool.

Another object of the present invention is to provide a laser handpiece which eliminates the eye discomfort caused to the user by the bright red aiming light while still providing visual feedback to indicate the laser energy delivery site.

Another object of the present invention is to provide a laser handpiece which enables the user to use the device for long periods of time without the discomfort caused by a hot handle.

The present invention comprises optical fibers held by friction fit in a handpiece comprised of two members:
1) a longitudinal sleeve composed of DELRIN polymer (or any substance with comparable mechanical and translucent properties) having a translucent distal end section for encasing an extendible laser fiber tip; and
2) a longitudinal sleeve whose proximal end has a smaller opening for cooperatively engaging the laser fiber in the friction fit holding the fiber within the sleeve at a desired distance from the distal end of the DELRIN polymer sleeve.

The two sleeves are mechanically connected (e.g. by threads as shown in FIG. 1) or optionally by a friction fit to form one cylindrical handpiece.

The laser fiber enters the handpiece proximally and it's position is easily maintained by friction rather than being fixed by a conventional gripping device as shown in the prior art drawing of FIG. 6. The DELRIN sleeve prevents eye irritation to the user caused by the bright red laser aiming beam and yet allows enough light to come through to see the location of the fiber's tip. Additionally, the DELRIN prevents heat build-up from the burning of tissue over a long period of time.

### DETAILED DESCRIPTION OF THE PREFERRED EMBODIMENT

Referring to FIG. 1, a laser fiber handpiece is pictured comprising an optical fiber encased within an optical fiber sheath (30) and a handpiece (1) comprising a longitudinal sleeve (10) proximally holding the fiber sheath (30) and a second longitudinal sleeve (20) distally exposing the fiber tip and mechanically fixed onto the first longitudinal sleeve (10). The proximal end (12) of the longitudinal sleeve (10) is externally tapered. The entire metal sleeve (10) including proximal end (12) is drilled to a diameter which gives a friction fit to the optical fiber sheath.

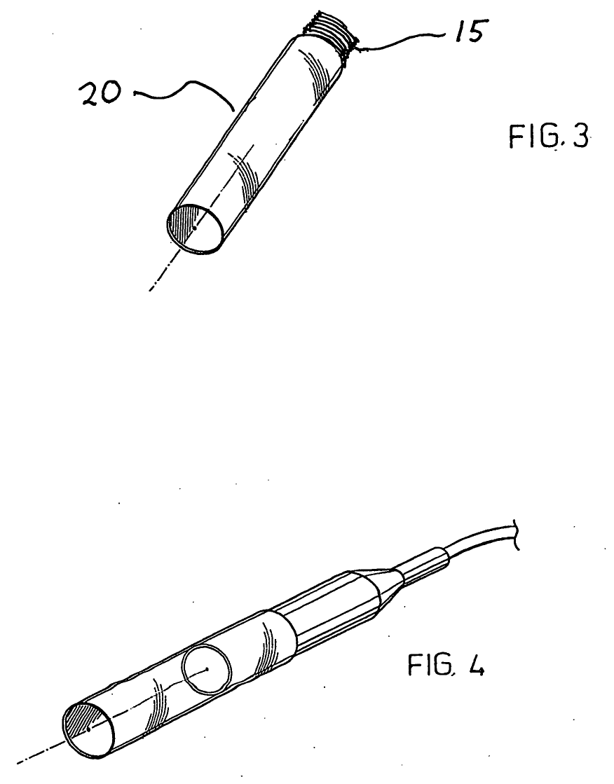

FIG. 3 illustrates the two sleeves, a metallic sleeve (10) and a DELRIN sleeve (20) separated,. The male threads (15) of the DELRIN sleeve are shown. The proximal sleeve (10) has the corresponding female threads (not shown). The two are connected together to form the combined handpiece.

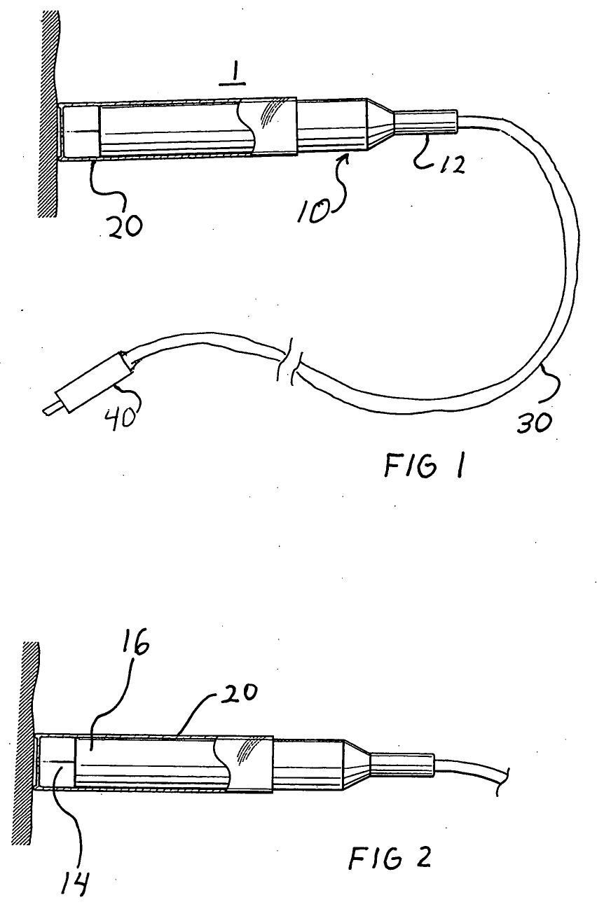

FIG. 2 again shows the two sleeves (10) and (20) as separate members with the female threads (16) of the sleeve (10), the inserted optical fiber sheath (14) held by friction fit and the distal end bore of the sleeve (20) are visible.

FIG. 4 shows the distal end bore of the sleeve (20) with the fiber (14) protruding into the bore.

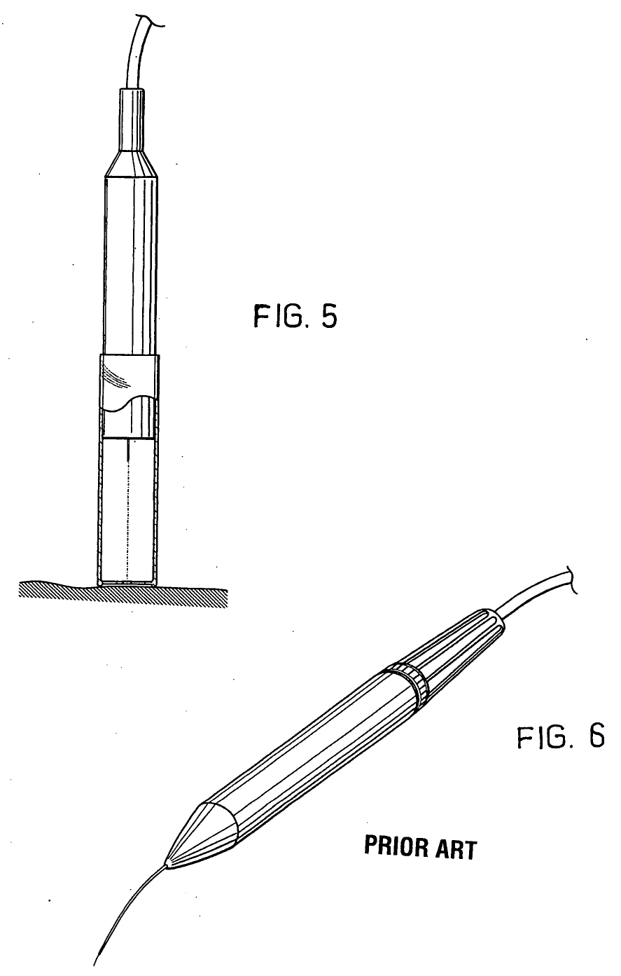

The assembled device in FIG. 1 can be connected to a conventional laser beam generator by means of the fiber connector (40). Then the user sets the position of the fiber into the handpiece by sliding it in or out to the desired depth. The user then turns on the generator and aims the laser energy emitted from the fiber onto the target. It is anticipated that the handpiece is held in close proximity to the target area as best shown in FIG. 5 and therefore the handpiece is inherently setting the minimum distance at which the fibers can be held away from the target.

It is further anticipated that the portion of the fiber shaft (32) just outside the proximal end of the handpiece (12) in FIG. 1, could be marked with numbered index marks to help the user to quickly set the fiber depth in the bore and to quickly check the depth.

It is further anticipated that the distal end of the DELRIN sleeve (20) could have longitudinal slits that could be exposed by sliding a cover moveably affixed to the sleeve to aid in setting the depth and in the guiding of the laser energy if desired. It is also contemplated that a dye or pigment could be used with other polymers to produce a sleeve having a translucent effect, or that a thin sleeve .of the translucent polymer could be utilized in combination with a sleeve comprised of another transparent or translucent material.

The anticipated us of this complete system is not limited to periodontal, tissue biostimulation or tissue debridement but is expected to be useful an any medical situation that uses laser energy exposed to living tissues.

Obvious benefits of the present invention are the inexpensive and rather simple parts involved and the ease, comfort and simplicity of use.

The foregoing detailed description is given primarily for clearness of understanding and no unnecessary limitations are to be understood therefrom, for modification will become obvious to those skilled in the art upon reading this disclosure and may be made upon departing from the spirit of the invention and scope of the appended claims. Accordingly, this invention is not intended to be limited by the specific exemplifications presented herein above. Rather, what is intended to be covered is within the spirit and scope of the appended claims.

We claim:

1. A laser fiber holder for exposing a laser energy to living tissue comprising:
a longitudinal sleeve comprising:
a translucent distal end sleeve portion for encasing an extendible laser fiber tip spaced apart from an interior bore sidewall thereof and energizable for biostimulation treatment of tissue and a proximal sleeve portion having a proximal end including a small opening sized for cooperatively engaging the insulated exterior surface of said laser fiber in a friction fit holding the fiber within the sleeve at a desired length and holding the tip at a set distance from the tissue providing said laser energy at a selected point above the tissue and protecting the eyes of the user from direct laser light.

2. The laser fiber holder of claim 1, wherein the longitudinal sleeve comprises a first translucent section having an inner bore spaced apart from said laser tip, said first translucent section co-operatively engaging a second sleeve section having an interior bore for cooperatively and slidably engaging said laser fiber insulated exterior surface in a friction fit.

3. The laser fiber holder of claim 2, wherein said second sleeve section comprises a heat resisting material.
